(19) United States
(12) Patent Application Publication
(10) Pub. No.: US 2014/0092215 A1
(43) Pub. Date: Apr. 3, 2014

(54) ENDOSCOPIC SYSTEM

(71) Applicant: Canon Kabushiki Kaisha, Tokyo (JP)

(72) Inventor: Akira Hayama, Yokohama-shi (JP)

(73) Assignee: Canon Kabushiki Kaisha, Tokyo (JP)

(21) Appl. No.: 14/021,558

(22) Filed: Sep. 9, 2013

(30) Foreign Application Priority Data
Oct. 2, 2012 (JP) 2012-220375

(51) Int. Cl. A61B 1/00; A61B 1/06

(52) U.S. Cl. CPC A61B 1/00045; A61B 1/0669
USPC 348/45

(57) ABSTRACT

In a stereoscopic endoscope apparatus, images obtained by left-eye and right-eye imaging systems are shaded differently due to subtle difference in an illumination direction, making fusion difficult. An endoscopic system includes: a stereoscopic endoscope which includes a light source of illumination light configured to illuminate inner part of a test object, an illumination window configured to emit the illumination light, and two or more imaging systems configured to image the inner part of the test object illuminated by the illumination light; and an illuminance distribution changing unit configured to change an illuminance distribution of the illumination light so as to reduce a difference in luminance distribution between/among pictures sensed by the respective imaging systems.

### BACKGROUND OF THE INVENTION

1. Field of the Invention

The present invention relates to an endoscopic system, and more particularly, to an endoscopic system including a controller configured to supply illumination light during imaging.

2. Description of the Related Art

An endoscope is widely used as a tool for observing inner part of a living body or a gap in a small space. Objects under observation through the endoscope are generally located in dark environments, and thus the endoscopic system is normally equipped with a light source of illumination light to illuminate the observed objects. If the illumination with the illumination light is uneven, some portions of the object will not be observed clearly. Therefore, with conventional endoscope apparatus, the illumination light are adjusted such that the object under observation is evenly illuminated. However, observed objects often contain areas with fine irregularities or areas with different surface conditions, whereas observations of such areas are often important in endoscopic observations. In view of the above, Japanese Patent No. 4714521 discloses a method for facilitating a diagnosis of a lesion by shading an observation region by causing a discrepancy between illuminance distributions of a pair of illumination lights emitted from a pair of illuminating units.

However, in the case of a stereoscopic endoscope apparatus made up of plural imaging systems, illumination directions and imaging directions of individual imaging systems differ subtly from each other. Furthermore, when a non-planar object such as an organ is observed with an endoscope, directivity is produced in reflected light due to irregularities of the observed object. Therefore, intensity of the reflected light received by each imaging system varies with an illumination window, areas of the object under observation, positional relationship between the imaging systems, and inclinations of the areas of the observed object. Consequently, in an image shot by each imaging system, differences occur in luminance among various areas of the imaged object under observation, making fusion difficult.

### SUMMARY OF THE INVENTION

The present invention has been made in view of the above problem and has an object to make fusion of images shot by plural imaging systems easier using a method which reduces differences in luminance among various areas caused by directivity of reflected light when an object under observation is illuminated from different positions and imaged at different positions.

In view of the above, the endoscopic system provided in the present invention includes: a stereoscopic endoscope which includes a light source of illumination light configured to illuminate inner part of a test object, an illumination window configured to emit the illumination light, and two or more imaging systems configured to image the inner part of the test object illuminated by the illumination light; and an illuminance distribution changing unit configured to change an illuminance distribution of the illumination light so as to reduce a difference in luminance distribution between/among pictures sensed by the respective imaging systems.

Further features of the present invention will become apparent from the following description of exemplary embodiments with reference to the attached drawings.

### BRIEF DESCRIPTION OF THE DRAWINGS

FIG. 1 is a functional block diagram of an embodiment of the present invention.

FIG. 2 is an example of images shot by respective imaging systems.

FIG. 3 is an example of an object under observation, illustrated physically, according to an embodiment of the present invention.

FIG. 4 shows non-illuminating regions used for illumination corresponding to a corrected illuminance distribution in an embodiment of the present invention.

FIG. 5 shows images shot by respective imaging systems under illumination corresponding to a corrected illuminance distribution in an embodiment of the present invention.

FIG. 6 shows images shot by respective imaging systems under illumination corresponding to a corrected illuminance distribution in an embodiment of the present invention.

FIG. 7 shows a distal part of a stereoscopic endoscope in Example 1.

FIG. 8 is an example of a physical object under observation in Example 1.

FIG. 9 shows images shot by respective imaging systems without applying a method according to the present invention in Example 1.

FIG. 10 shows a non-illuminating region used for illumination corresponding to a corrected illuminance distribution in Example 1.

FIG. 11 shows images shot by respective imaging systems by the application of the method according to the present invention in Example 1.

FIG. 12 is a schematic diagram of a light source device in Example 1.

FIG. 13 shows images shot by respective imaging systems without applying the method according to the present invention in Example 2.

FIG. 14 is an example of a physical object under observation in Example 2.

FIG. 15 shows a non-illuminating region used for illumination corresponding to a corrected illuminance distribution in Example 2.

FIG. 16 shows images shot by respective imaging systems by the application of the method according to the present invention in Example 2.

FIG. 17 shows images shot by respective imaging systems without applying the method according to the present invention in Example 3.

FIG. 18 shows non-illuminating regions used for illumination corresponding to a corrected illuminance distribution in Example 3.

FIG. 19 shows images shot by respective imaging systems by the application of the method according to the present invention in Example 3.

FIG. 20 is a structural diagram of a distal part of an endoscope in Example 4.

FIG. 21 shows images shot by respective imaging systems without applying the method according to the present invention in Example 4.

FIG. 22 shows non-illuminating regions used for illumination corresponding to a corrected illuminance distribution in Example 4.

### DESCRIPTION OF THE EMBODIMENTS

An exemplary embodiment of the present invention will be described in detail with reference to the accompanying drawings. However, the scope of the present invention is not limited to the illustration examples.

FIG. 1 shows a functional block diagram of the present embodiment. At a distal end of an endoscope inserted inside a test object, an endoscopic system according to the present invention has a right-eye imaging system 101R and left-eye imaging system 101L for a stereoscopic endoscope. Also, the endoscopic system includes a memory 11, an image processing unit 12, and a light source 13. The light source 13 can emit light to illuminate a fixed coverage, and can change illuminance depending on an area within an illumination coverage, using a configuration described later. A system made up of two imaging systems—for the right eye and the left eye—will be described herein, but the number of imaging systems is not limited to this, and the endoscopic system of the present invention can have two or more imaging systems.

Also, the endoscopic system according to the present invention may include a fusion processor (not shown) configured to fuse images sensed by the respective imaging systems and a display (not shown) configured to display the fused images.

With this configuration, the images captured by the imaging systems 101R and 101L are held temporarily in the memory 11. The image processing unit 12 calculates luminance distributions of imaging regions from the images held in the memory 11 and conveys a corrected illuminance distribution to the light source 13 based on resulting information. The light source 13 illuminates the object under observation using an illuminance distribution corrected based on the conveyed information.

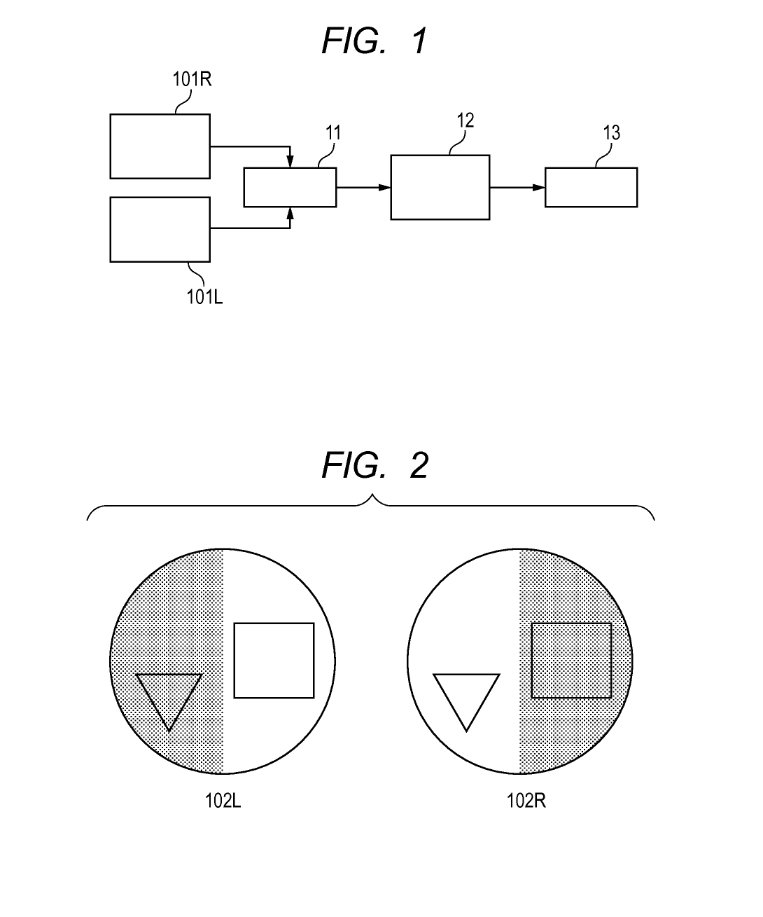

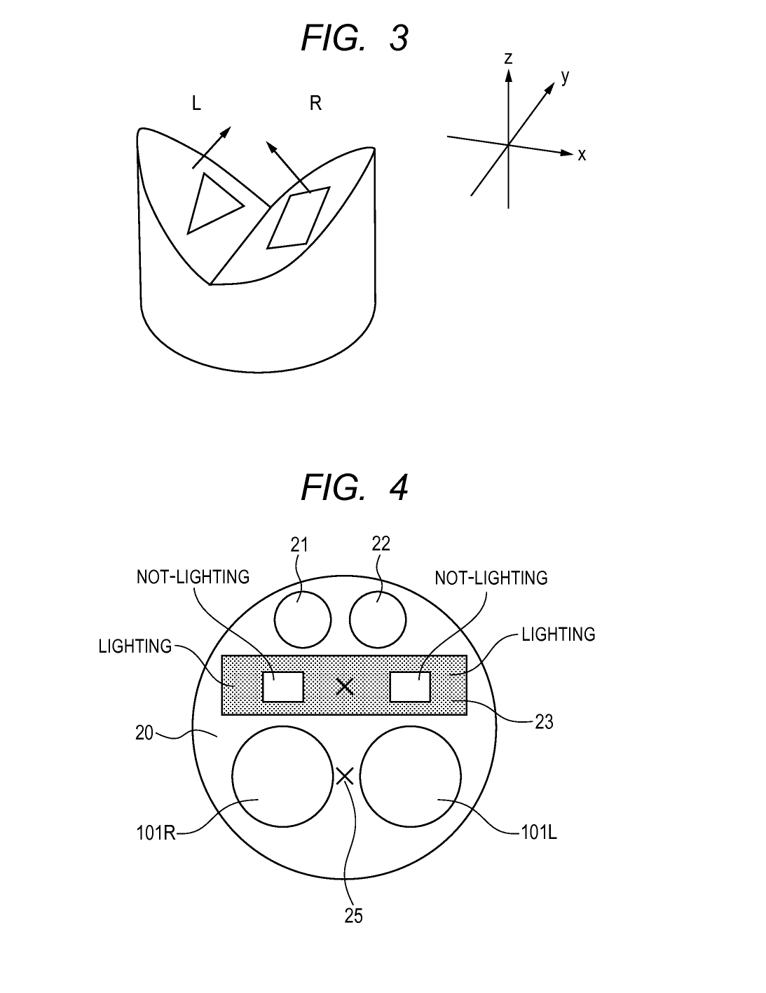

FIG. 2 is an example of images 102R and 102L saved in the memory 11 when a shape depressed from left and right toward a center of an observation region such as shown in FIG. 3 is shot by the imaging systems 101R and 101L, respectively, placed in a z direction of the object under observation in FIG. 2. In so doing, the light source 13 is located in the z direction in FIG. 2 when viewed from the object under observation as in the case of the imaging systems 101R and 101L. That is, the lighting direction coincides with the viewing direction. In that region of an observation site in which an imaging system is located in the direction of a reflection angle as opposed to an incident angle of the illuminating light, the imaging system directly receives reflected light, resulting in a relatively high luminance in a sensed image while other regions produce a relatively low luminance. Consequently, in FIG. 2, the regions directly opposite the respective imaging systems, i.e., the left half region of the image 102R and right half region of the image 102L, have high luminance. In this way, if there are differences in the luminance of some regions (according to the present invention, this is also described as there being differences in luminance distribution) between images, the images do not mix properly when fused, and the object under observation might become indistinct. Thus, according to the present embodiment, the image processing unit 12 serving as a luminance difference determination unit determines the luminance of each imaging area obtained by dividing the sensed images into areas of a fixed size (a fixed number of pixels), and determines whether the luminance of any of the imaging area in each image is obviously high. Furthermore, the image processing unit 12 serving as an illuminance distribution changing unit provides a corrected illuminance distribution to the light source 13 so as to reduce the luminance of the imaging area determined as having high luminance.

Specifically, when an average luminance value of an imaging area obtained by dividing the sensed picture is equal to or larger than a predetermined value, the image processing unit 12 serving as a luminance difference determination unit determines that the imaging area obviously has a high luminance. In so doing, the effects of the present invention can be achieved, for example, if it is determined that the imaging area obviously has a high luminance when the average luminance value of the imaging area is equal to or higher than 80% or 90% the maximum luminance value of the screen. Also, when the average luminance value of each imaging area obtained by dividing the sensed picture is compared with the average luminance value of the corresponding imaging area in a picture sensed simultaneously by another imaging system, if the absolute value of the difference in the average luminance value between an imaging area and the corresponding imaging area is equal to or larger than a predetermined value, it may be determined that the imaging area in the picture with the higher luminance obviously has a high luminance. In so doing, the effects of the present invention can be achieved, for example, if it is determined that the imaging area in the picture with the higher luminance obviously has a high luminance when the difference in the average luminance value between the imaging areas is equal to or higher than 10% or 20% the maximum luminance value of the screen. Note that the size of the imaging areas and method of division as well as the average luminance value, the absolute value of the difference in the average value, and other criteria for determining luminance as obviously being high can be set freely according to the purpose of observation and the object under observation.

Because the high-luminance portion is produced by reflected light made directly incident upon the imaging system by the shape of an organ surface, the illuminance distribution can be adjusted so as to lower the illuminance of the illuminating light in the region which produces the reflected light.

Specifically, according to the present embodiment, the image processing unit 12 serving as an illuminance distribution changing unit stores an illumination window region whose illumination intensity needs to be changed in order to reduce the luminance of each imaging area, by associating the illumination window region with distances of the object from the illumination window as well as from the imaging system, and then retrieves the illumination window region whose illumination intensity needs to be changed in order to reduce the image luminance distribution in each imaging area. The distance between the imaging system and object can be found from stereoscopy information, and specifically, from an amount of disparity, but a distance sensor may be installed or the distance may be substituted with a typical imaging distance. Furthermore, the image processing unit 12 serving as an illuminance distribution changing unit calculates how to change the intensity of light emitted through the retrieved illumination window region, i.e., the intensity of the emitted light after the change, based on differences in the average luminance value among imaging areas. By correcting the illuminance distribution in this way, the illuminance distribution can be corrected according to the luminance distribution of the sensed image so as to produce a luminance distribution which allows easy fusion not only in the examples of FIGS. 2 to 6.

If the luminance has been made wholly low by the above process, the illuminance distribution change can bring overall luminance to an appropriate level by increasing the illuminance on average so as to increase overall luminance.

Specifically, according to the present embodiment, the image processing unit 12 serving as an illumination light intensity changing unit determines the average luminance value of the entire image after the illuminance distribution is changed by the illuminance distribution changing unit. Then, if the average value is equal to or smaller than a predetermined value, the image processing unit 12 increases the illuminance such that an equal ratio will be obtained in the entire illumination range without changing a pattern of the changed illuminance distribution. In so doing, the effects of the present invention can be achieved, for example, if it is determined that the luminance of the screen is low as a whole when the average luminance value of the entire image is equal to or lower than 10% or 20% the maximum luminance value of the apparatus.

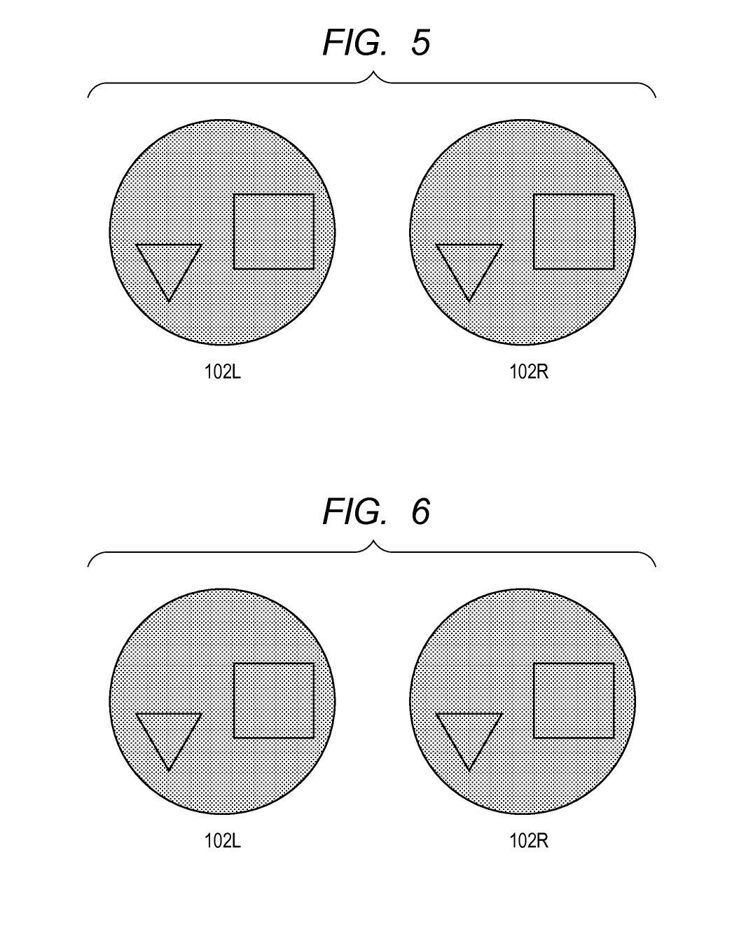

According to the present embodiment, the corrected illuminance distribution is changed such that the right half of the image 102L and left half of the image 102R will be dark. In this case, to reduce the reflected light received directly from the object by each imaging system, part of the illuminating light is unlit as shown in FIG. 4. Regarding the corrected illuminance distribution, although lit and unlit regions may be provided in the light source 13 as described above, similar effects can be obtained, for example, by producing an intensity distribution in the illuminance of the illuminating light from the light source 13. The use of the corrected illuminance distribution for illumination reduces differences in luminance distribution as shown in FIG. 5 and makes fusion easier. Overall luminance will decrease if nothing is done, but can be increased by slightly increasing overall illuminance as shown in FIG. 6.

Note that the primary objective of the present invention is not to eliminate (smooth) luminance distributions in sensed images. As described later in examples, as long as differences in luminance distribution between the images sensed by different imaging systems can be reduced, high-luminance portions produced by the illumination light may be left in the sensed images.

The illumination of the endoscope apparatus operates by being emitted from the illumination window at a distal end of an insertion portion of the endoscope via a light guide housed in the endoscope connected to the light source 13. The illumination window does not have a definite shape in particular, and may have a circular, triangular, square or appropriately curved shape. The shape may be determined depending on positional relationship with other components. From the standpoint of individual optimization, each imaging system may have an independent illumination window. As an illumination light source, a high-luminance, high-voltage discharge tube such as a xenon lamp, metal halide lamp or halogen lamp can be used. The light guide is made up of plural fiber optic bundles, and it is advisable that incident light distribution on the fiber optic bundles coincides with the illuminance distribution.

A number of lenses as well as an optical modulation device configured to limit the illumination light from the light source may be installed between the light source and light guide. As the optical modulation device, an electrical device such as a liquid crystal panel may be used, allowing the illuminance distribution to be changed more easily without limitation, but a mechanical device such as a diaphragm mechanism may be used as well. With an electrical device such as a liquid crystal panel, the illuminance distribution can be changed by changing the light quantity of the illumination light emitted from part of the illumination window. Also, when light-emitting diodes (LEDs) are used as the light source, the light distribution can be controlled by regulating the intensity of light from the light source without using an optical modulation device.

Besides, the possibility of high-luminance portions being produced can also be reduced by forming a shield wall on the illumination window at a distal end of an insertion portion of the endoscope, thereby preventing increase in the coverage of the illumination light emitted from the illumination window and narrowing the region from which the illumination light is reflected.

As described so far, stereoscopic endoscopic images which readily lend themselves to fusion can be provided if differences in luminance distribution between the images sensed by different imaging systems are detected, thereby changing the illuminance distribution so as to correct the differences and thereby reducing the differences in luminance distribution. Incidentally, since it is conceivable that a luminance distribution will be changed by a corrected illuminance distribution, resulting in new differences in luminance distribution, if corrections to illuminance distribution such as described above are made multiple times, differences in luminance distribution can be reduced more reliably. If the right-eye imaging system and left-eye imaging system are made to substantially coincide in an illumination direction or if the illumination direction made to substantially coincide between the imaging systems is set appropriately, contrast or shadows on a subject are enhanced and irregularity patterns and surface conditions become distinctive, making fusion, i.e., stereoscopy, still easier.

### EXAMPLES

The present invention will be described in detail below with concrete examples.

### Example 1

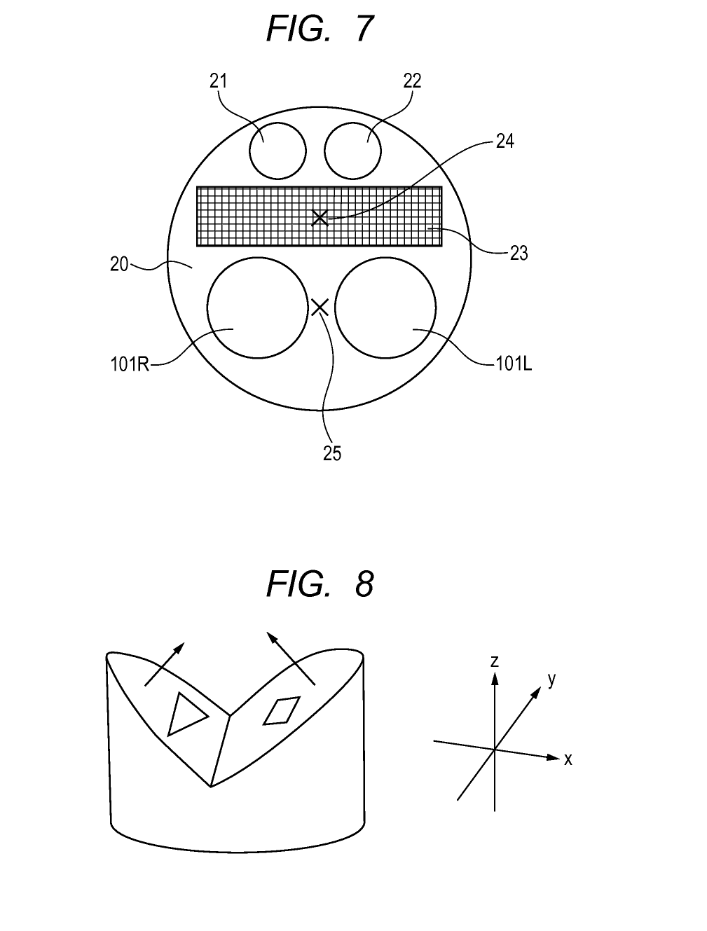

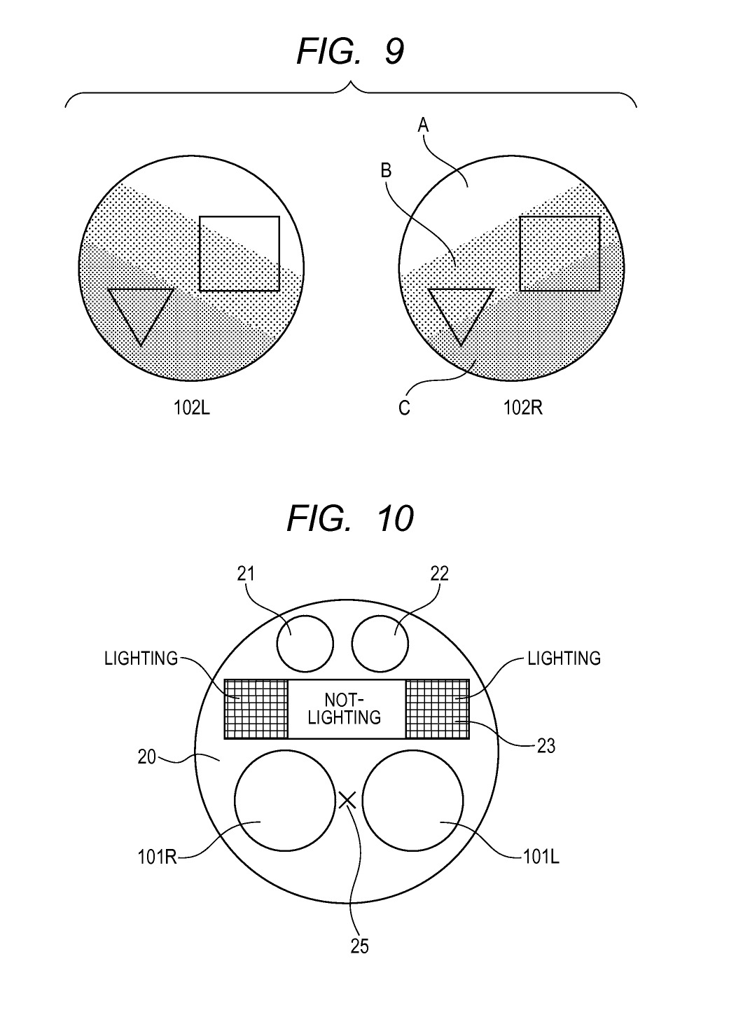

A distal part 20 of a stereoscopic endoscope in this example shown in FIG. 7 had channel holes 21 and 22 and an illumination window 23. In this example, the diameter of the endoscope was 10 mm, the diameter of the imaging systems 101R and 101L was 3 mm, the viewing direction angle was 70 degrees, the diameter of the channel holes 21 and 22 was 1.5 mm, and the illumination window was 1.5 mm high×8 mm wide. Also, an aperture angle (2θ) of an optical fiber used for illumination was 20 degrees. The endoscope was placed such that the imaging systems and illumination would be oriented in the z direction of an object under observation in FIG. 8, where the object was depressed from left and right toward a center of an observation region with the depression being inclined toward the near side, such as shown in FIG. 8. The object was observed from the near side without applying the present invention, and images obtained by the imaging systems 101R and 101L inserted through the channel holes are shown in FIG. 9. A lens-to-object distance was 5 mm. In FIG. 9, each observation image is divided into three regions A, B, and C according to the luminance for the sake of convenience. The luminance of A, B, and C decreases in the order A>B>C. In FIG. 9, in addition to the difference in luminance between the left and right of the image such as also shown in FIG. 2, a luminance distribution occurs also in a vertical direction of the image due to inclination of the object under observation in the vertical direction of the image, and consequently a gradient of the luminance distribution occurs in an oblique direction in the image as a whole. In this example, since the object under observation is depressed from left and right toward the center, the luminance distributions in the image 102R and image 102L are axisymmetric with respect to a line (axis) passing through the center 24 of the illumination window and the midpoint 25 between the imaging systems 101R and 101L as shown in FIG. 9. Therefore, even if one attempts to fuse the image 102R and image 102L by superimposing the images 102R and 102L on each other as they are, the luminance varies between the superimposed portions, making fusion difficult and resulting in a difficult-to-observe image.

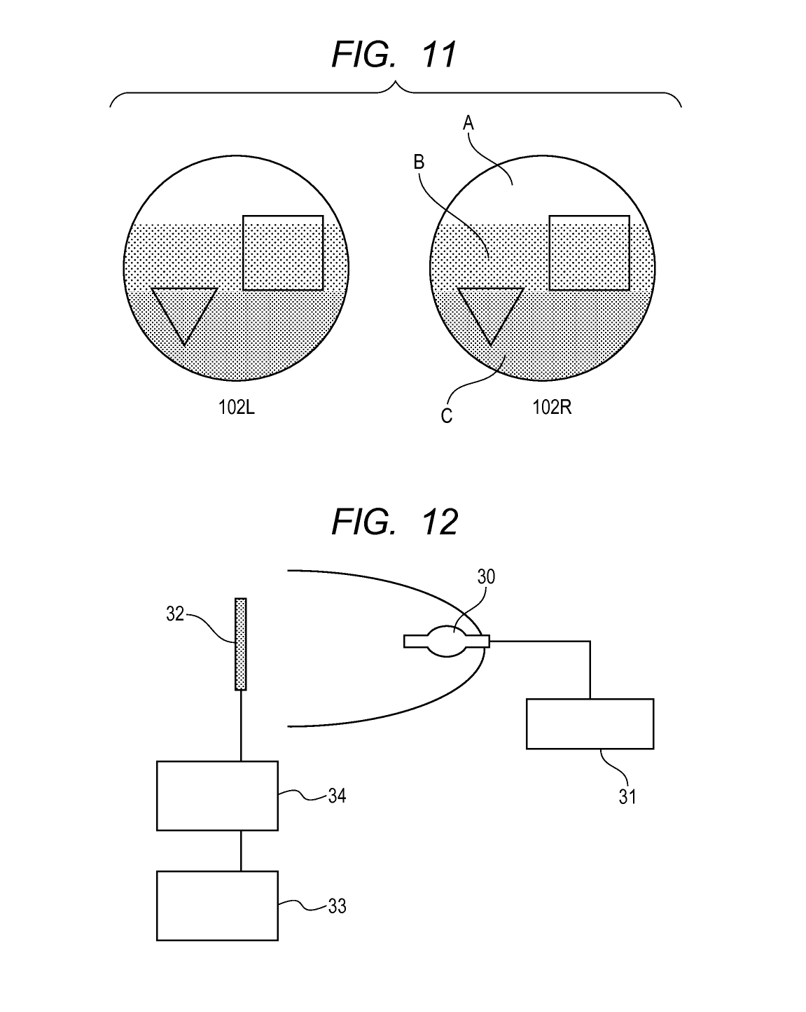

FIG. 11 shows observation images obtained when a 4-mm central portion of illumination was kept unlit as in FIG. 10. Although the luminance of images still decreased in the order A>B>C due to inclination of the object under observation in the vertical direction of the images, the image 102R and image 102L had similar luminance distributions with horizontal gradients of the luminance distributions being curbed in both the images. The reason for this is as follows: in the case of the image 102L, for example, since the center of illumination was kept unlit, reflected light entering the imaging system 101L, i.e., the light emitted from the center of illumination and specularly reflected off the upper right region of the sensed image, was able to be reduced, the influence of the distribution of high-luminance portion centered at the upper right portion of the image 102L was reduced, resulting in reduced horizontal gradients of the luminance distributions. Similarly, in the case of the image 102R, by reducing the intensity of the reflected light entering the imaging system 101R after being reflected off the upper left region of the sensed image, the horizontal gradients of the luminance distributions were reduced. In this way, since the horizontal gradients of the luminance distributions are reduced in both the images 102R and 102L, reducing the horizontal differences in luminance distribution between the images 102R and 102L as well, when the images 102R 102L are fused by being superimposed on each other as they are, the superimposed portions are equal in luminance distribution, and thus the resulting image is not difficult to observe.

As shown in FIG. 12, the light source device in this example included a Halogen light source 30 serving as a light source lamp configured to emit illumination light, a lamp power supply 31 for the light source, and a liquid crystal panel 32 installed in front of the light source and serving as the optical modulation device configured to control the light distribution by limiting transmitted light quantity of the illumination light, as well as a liquid crystal control circuit 33 and a liquid crystal drive circuit 34 configured to control and drive a pattern of the liquid crystal panel, respectively, based on settings specified by the image processing unit 12. Desirably the liquid crystal panel is a monochrome panel such as used for a data projector, and a panel made up of 1024×768 elements was prepared in this example. Using the light source device configured as described above, illumination control such as described above was performed by changing the transmittance of the liquid crystal panel by means of the liquid crystal control circuit 33.

As described above, stereoscopic endoscopic images which readily lend themselves to fusion were able to be provided by detecting differences in luminance distribution between the image shot by the imaging system 101R and image shot by the imaging system 101L, emitting light according to such an illuminance distribution as to correct the differences, and reducing the differences in luminance distribution.

### Example 2

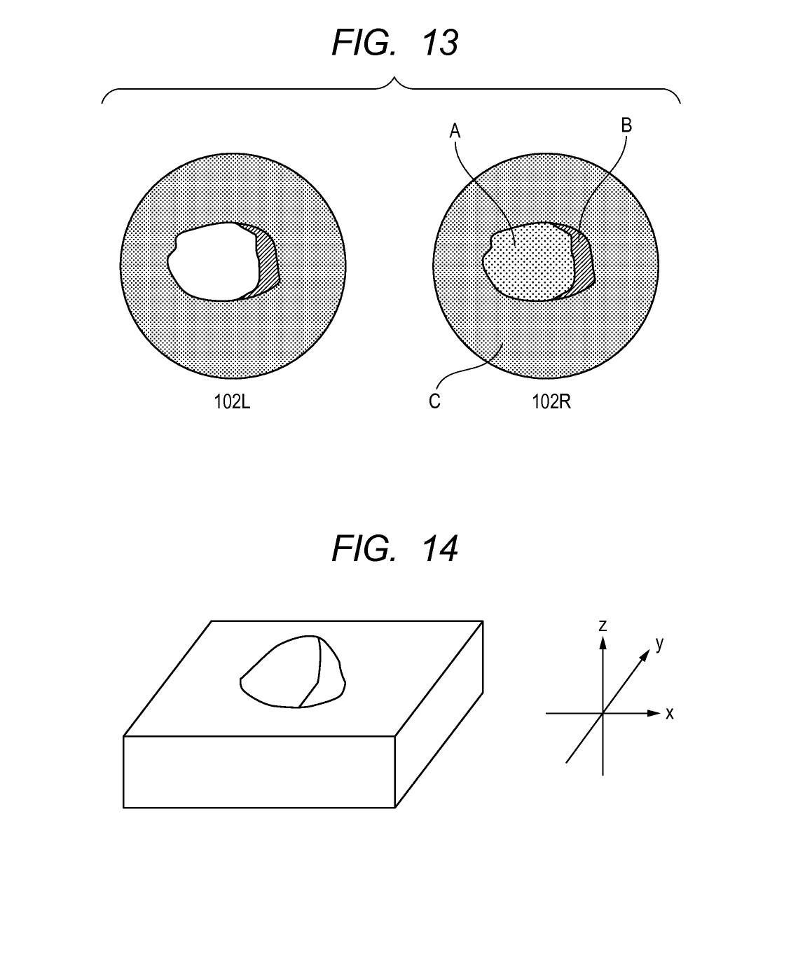

In this example, a specific area was processed according to the form of the subject. FIG. 13 shows an example of images obtained by observing an object from above through the same endoscope as in Example 1 using a light distribution such as shown in FIG. 7, the object under observation having a protrusion in part of its regions such as shown in FIG. 14. When brightness of regions A, B and C are compared between the image 102R and image 102L, it can be seen that only region A is brighter in the image 102L than in the image 102R whereas both regions B and C are almost equal in brightness between the two images. This is because the circumstance is such that reflected light from region A of the subject enters only the imaging system 101L. In this way, if there are differences in the luminance of some regions between images, the images do not mix properly when fused, and the object under observation might become indistinct.

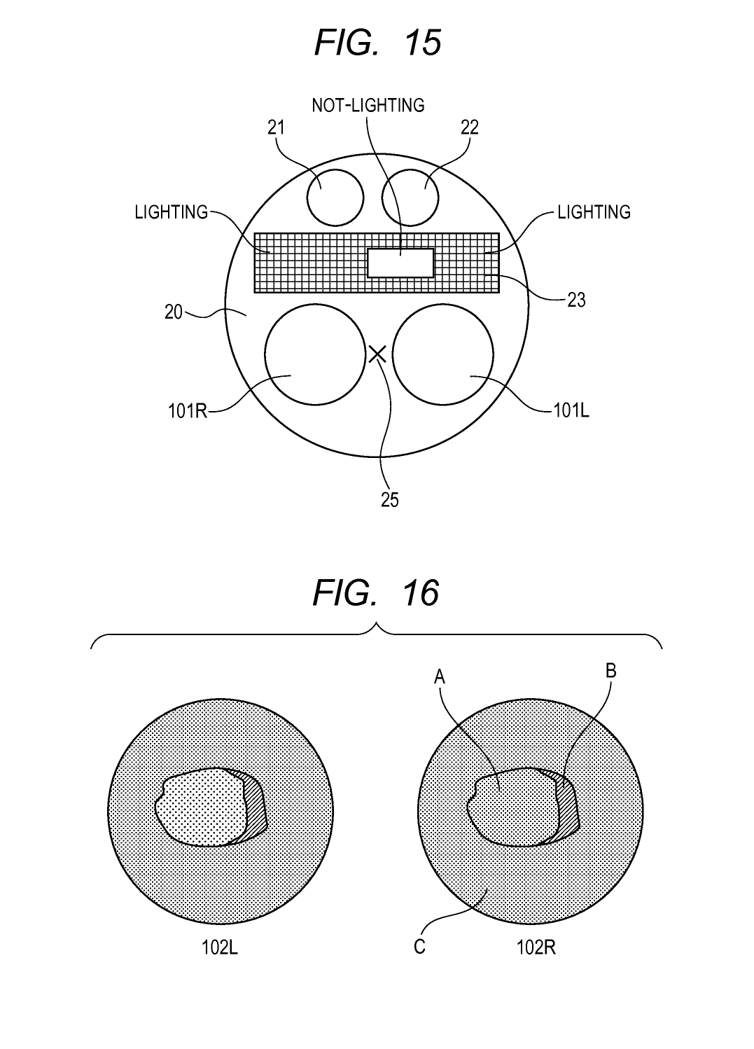

In this example, by reducing directly reflected light from region A in the imaging system 101L under lighting conditions such as shown in FIG. 15, observation images such as shown in FIG. 16 can be obtained. The lighting conditions shown in FIG. 15 are established as follows. First, the image processing unit 12 recognizes that region A of the image 102L is brighter than region A of the image 102R from the observation images, creating a luminance difference. Then, the image processing unit 12 serving as an illumination light intensity changing unit determines from what region of illumination the illuminating light incident upon region A is mainly emitted, based on a precalculated pattern of illuminance distribution and light output regions. In this example, spread of illumination from each light output region was ignored, and thus the illuminance distribution was unchanged even if distance changed. However, light output regions may be determined by taking into consideration the spread of illumination from the light output regions and distance to the subject. When the region whose illumination light is to be changed is definitely determined, an amount of intensity change is determined based on the luminance difference between region A of the image 102L and region A of the image 102R. Specifically, adjustments are made such that region A of the image 102L will become equal in brightness to region A of the image 102R. This reduces the difference in luminance distribution between the image 102L and image 102R, providing observation images with a reduced difference in luminance distribution such as shown in FIG. 16.

### Example 3

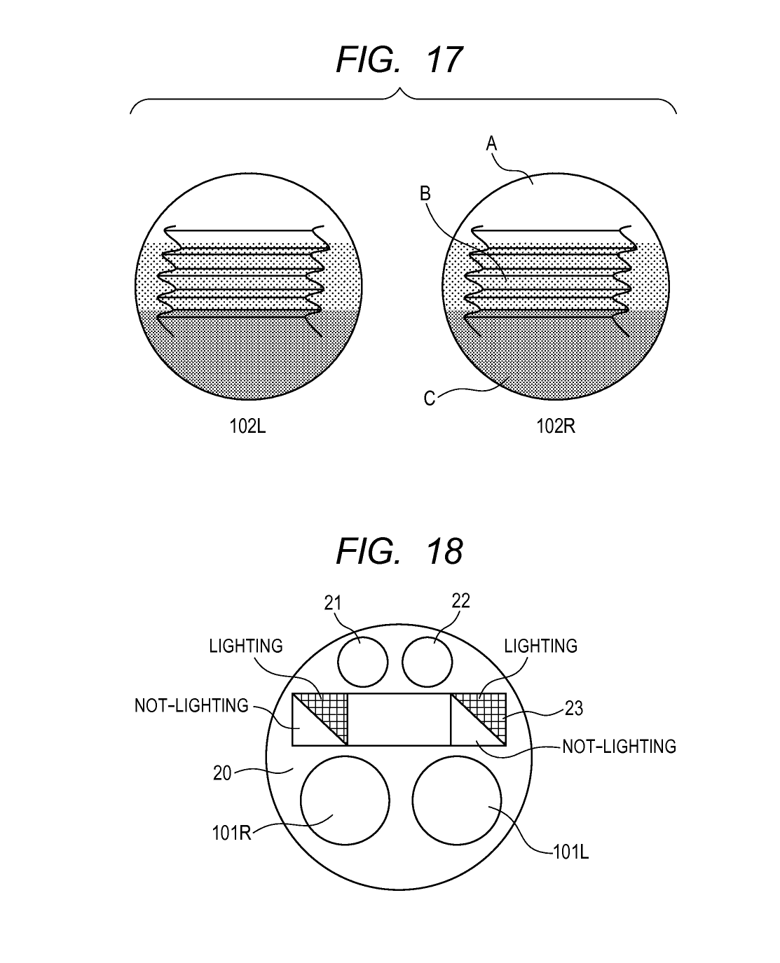

This example relates to a mode of selecting a light distribution according to a form, surface conditions or the like of the subject. FIG. 17 shows an example of observation images obtained by observing an object through the same endoscope as in Example 1 using a light distribution such as shown in FIG. 10, the object under observation being depressed from left and right toward the center of an observation region inclined forward on the plane of the paper as in the case of Example 1. However, in this example, on the surface of the object under observation, a wavy irregularity structure was provided in the horizontal direction of the observation region. As shown in FIG. 17, the luminance distribution was almost the same between the image 102R and image 102L. However, in the tendency of luminance distribution, i.e., in the horizontal direction of the screen, the luminance distribution pattern coincided with a structural feature of the subject, i.e., the pattern of the wavy structure spreading in the horizontal direction of the screen, making the structure of the subject indistinct.

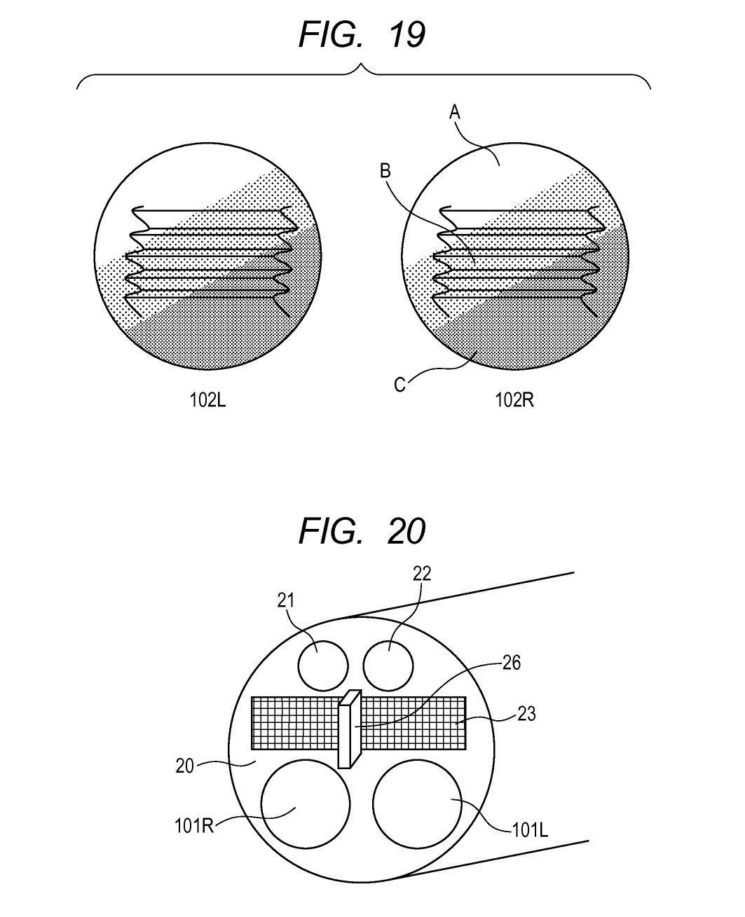

In this example, the use of the lighting conditions shown in FIG. 18 allowed the illumination direction to be changed based on the illumination distribution, thereby providing observation images such as shown in FIG. 19. At this time, in the original images, an irregularity structure may appear as a simple pattern and the existence thereof may not be distinguished. In such a case, if the illumination direction is changed slightly, the irregularity structure may become easier to distinguish. In this example, if the illumination direction is rotated approximately 45 degrees to the right, the irregularity structure becomes easier to distinguish. Regarding the rotation of the illumination direction, an optimum angle can be selected by precalculating an illumination light intensity distribution needed to rotate an illumination light pattern in 15-degree increments and trying out the rotated illumination directions a few times. In this way, if the horizontal gradients of the luminance distributions are adjusted in the images 102R and 102L according to surface geometries of the object under observation and the direction of gradations in the illuminance distribution is shifted to the direction of the irregularity structure so as to shade the irregularity structure, the structure of the subject can be made easier to see. Note that the image 102R and image 102L do not need to coincide completely in the distribution of luminance and direction of illumination.

### Example 4

In this example, a shield wall was installed on the illumination window of the same endoscope as Example 1 to increase controllability of the illuminance distribution.

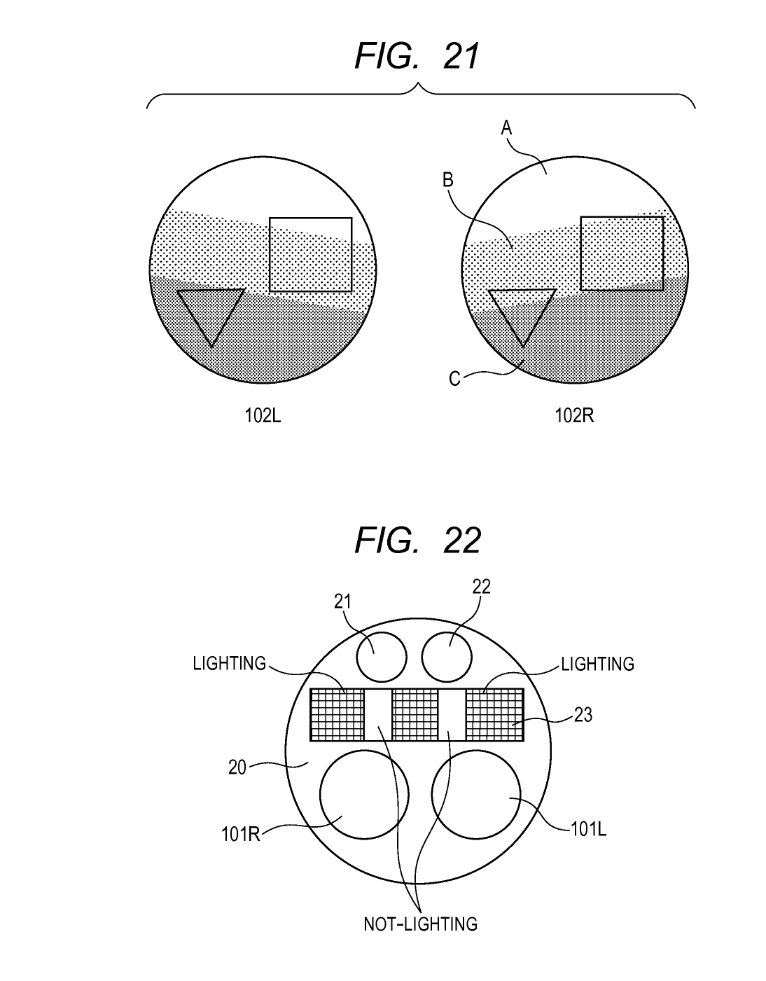

FIG. 20 illustrates a distal part of an endoscope provided with a shield wall 26. In this example, the shield wall measured 1.7 mm long by 0.2 mm wide by 0.8 mm high. Other structures had the same dimensions as in Example 1. Observation images obtained by application of illumination based on a corrected illuminance distribution in the present example are shown in FIG. 21. Compared to the observation images in FIG. 9 obtained in Example 1 without applying the present invention, it can be seen that the differences in luminance distribution between the image 102R and image 102L were reduced. This is because the shield wall shielded emitted light oriented so as to produce reflected light with high directivity directed toward the imaging systems and thereby reduced the differences in luminance intensity among image regions in each of the image 102R and image 102L. In this state, illumination based on the corrected illuminance distribution was applied. Specifically, as shown in FIG. 22, two 1-mm wide unlit portions were provided in a central portion. Then, it was confirmed that differences in luminance distribution between the image 102R and image 102L were reduced as in the case of Example 1 in FIG. 11. This indicates that differences in luminance distribution between the image 102R and image 102L were able to be decreased in this example even though the luminance was reduced in a smaller region of the illumination than in Example 1 because the luminance distribution had been reduced by the shield wall from the beginning.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

By adjusting the illuminance distribution (including the illumination direction) used to illuminate a subject, the present invention decreases differences in the luminance of reflected light between observation positions when observed from the imaging positions of the respective imaging systems, making fusion, i.e., stereoscopy, through the endoscope easier.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-220375, filed Oct. 2, 2012, which is hereby incorporated by reference herein in its entirety.

What is claimed is:

1. An endoscopic system comprising:
a stereoscopic endoscope which includes:
a light source of illumination light configured to illuminate inner part of a test object,
an illumination window configured to emit the illumination light, and
two or more imaging systems configured to image the inner part of the test object illuminated by the illumination light;
an illuminance distribution changing unit configured to change an illuminance distribution used in illuminating the inner part of the test object, so as to reduce a difference in luminance distribution between/among two or more pictures sensed by the two or more imaging systems;
a fusion processor configured to fuse images sensed by the respective imaging systems; and
a display configured to display the fused images.

2. The endoscopic system according to claim 1, further comprising a determination unit configured to find a difference in an average luminance value of each imaging area between/among the images sensed by the two or more imaging systems and then determine that the luminance difference of the imaging area is large when the luminance difference is equal to or larger than a predetermined value, wherein
the illuminance distribution changing unit changes the illuminance distribution of the illumination light so as to reduce the luminance of the imaging area determined as having the large luminance difference, in the image with the larger luminance.

3. The endoscopic system according to claim 1, further comprising a determination unit configured to find an average luminance value of each imaging area in the images sensed by the imaging systems and then determine that the luminance of the imaging area is large when the average luminance value is equal to or larger than a predetermined value, wherein
the illuminance distribution changing unit changes the illuminance distribution of the illumination light so as to reduce the luminance of the imaging area determined as having the large luminance.

4. The endoscopic system according to claim 1, wherein the illuminance distribution changing unit stores an illumination window region whose illumination intensity needs to be changed in order to reduce the luminance of each imaging area by associating the illumination window region with distances from the illumination window and the imaging system to that area of the object which corresponds to the imaging area, and then calculates the illumination window region whose illumination intensity needs to be changed in order to reduce the luminance of the imaging area as well as emitted light intensity after the change, based on the difference in the luminance distribution between/among the images.

5. The endoscopic system according to claim 1, further comprising an illumination light intensity changing unit configured to increase intensity of the illumination light without changing the changed illuminance distribution of the illumination light if an average luminance value of the whole images after the change in the illuminance distribution is equal to or smaller than a predetermined value.

6. The endoscopic system according to claim 1, wherein the illuminance distribution changing unit further changes the illuminance distribution of the illumination light so as to shade irregularities on an object under observation.

7. The endoscopic system according to claim 1, further comprising a shield wall configured to shield part of the illumination light emitted through the illumination window.

8. The endoscopic system according to claim 1, wherein the illuminance distribution changing unit changes the illuminance distribution of the illumination light by changing light quantity of the illumination light emitted through part of the illumination window.

9. The endoscopic system according to claim 8, wherein the illumination window is a liquid crystal panel and the illuminance distribution changing unit changes transmittance of part of the panel.

10. The endoscopic system according to claim 8, wherein the illuminance distribution changing unit shields part of the illumination light emitted through the illumination window using a diaphragm mechanism.

11. The endoscopic system according to claim 8, wherein the light source includes a light-emitting diode and the illuminance distribution changing unit adjusts intensity of light coming from the light source.

12. An imaging method for an endoscope, comprising:
illuminating inner part of a test object;
imaging the inner part of the test object using two or more imaging systems, the inner part of the test object being illuminated by illumination light; and
changing an illuminance distribution used in illuminating the inner part of the test object, so as to reduce a difference in luminance distribution between/among two or more pictures sensed by the two or more imaging systems.
